(19) United States
(12) Patent Application Publication
(10) Pub. No.: US 2024/0139316 A1
(43) Pub. Date: May 2, 2024

(54) USE OF OREGOVOMAB AND BEVACIZUMAB, PACLITAXEL, CARBOPLATIN FOR TREATMENT OF BRCA-WILD TYPE PLATINUM SENSITIVE RECURRENT

(71) Applicant: CANARIABIO INC., Pyeongtaek-si (KR)

(72) Inventor: Ragupathy MADIYALAKAN, Edmonton (CA)

(21) Appl. No.: 18/280,981

(22) PCT Filed: Mar. 9, 2022
(86) PCT No.: PCT/CA2022/050347

§ 371 Date: Sep. 8, 2023

Related U.S. Application Data
(60) Provisional application No. 63/159,013, filed on Mar. 10, 2021.

(51) Int. Cl. A61K 39/395; A61K 31/282; A61K 31/337; A61K 45/06; A61P 35/00

(52) U.S. Cl. CPC A61K 39/39558; A61K 31/282; A61K 31/337; A61K 39/3955; A61K 45/06; A61P 35/00; A61K 2039/54

(57) ABSTRACT

The present document describes methods for improving likelihood of survival in a stage III-IV ovarian cancer patient, and the use of chemotherapeutic agents (paclitaxel and carboplatin) and therapeutic monoclonal antibodies (oregovomab and bevacizumab) combinations, for inhibiting stage III-IV ovarian cancer tumor growth in a patient. This combination of chemotherapeutic agents and therapeutic monoclonal antibodies is administered according to specific schedules involving 6 cycles of chemotherapy where in each cycle the patient is administered paclitaxel, carboplatin and bevacizumab on the same day, antibody mAb-B43.13 (oregovomab) concurrently during cycles 1, 3, and 5 of the 6 cycles of chemotherapy, and a final dose of monoclonal antibody mAb-B43.13 without concurrent chemotherapy about 10 to about 14 weeks after cycle 5 of the 6 cycles of chemotherapy.

### CROSS-REFERENCE TO RELATED APPLICATIONS

This application claims priority of U.S. provisional patent application No. 63/159,013 filed on Mar. 10, 2021, the specification of which is hereby incorporated by reference in its entirety.

### BACKGROUND

### (a) Field

The subject matter disclosed generally relates to methods for improving likelihood of survival in a stage III-IV ovarian cancer patient, and more particularly to methods for improving likelihood of survival in a stage III-IV ovarian cancer patient with a treatment involving paclitaxel, oregovomab (mAb-43.13), carboplatin and bevacizumab.

### (b) Related Prior Art

Oregovomab is an intravenously administered monoclonal antibody (MAb-B43.13) that binds the tumor associated CA125 antigen (also known as MUC16). Immune complexes of Oregovomab and CA125 are taken up by dendritic cells (DCs), leading to antigen presentation via MHC II as well as MHC I and activation of activation of CA125 specific CD4 Helper T cells, a humoral response and activation of CA125 specific CD8 Cytotoxic T cells. Front line chemo-immunotherapy with four infusions of Oregovomab with standard six cycle carboplatin and paclitaxel resulted in significant improvement in both progression free survival (PFS) and overall survival (OS) relative to carboplatin-paclitaxel alone (Ferrandina, Journal of Clinical Oncology 2017).

However, compared to the combination of paclitaxel and carboplatin, bevacizumab combination therapy with paclitaxel and carboplatin is the most effective current standard of care in advanced ovarian cancer as first and recurrent therapy, especially when focusing on BRCA-wild type ovarian cancer patients without DNA repair pathway defects, according to Gynecologic Oncology Group (GOG) phase 3 clinical trials. (Oza et al. Lancet Oncology 2015).

In addition, compared to PARP inhibitor which was a maintenance therapy only for patients who had responded to platinum-based chemotherapy, the combination of bevacizumab with paclitaxel and carboplatin increased the rate of response and rapidly improved symptoms associated with cancer progression or relapse. Thus bevacizumab combination therapy still plays an important role as first or recurrent therapy (Ray-Coquard, NEJM 2019).

Bevacizumab (Avastin™) is a recombinant humanized IgG1 monoclonal antibody (A4.6.1) that selectively binds to all isoform of human VEGF-A and neutralizes VEGFF's biological activity through blocking of the binding of VEGF to VEGFR1 and VEGFR2. Bevacizumab also improves immune microenvironment by recruitment immune effector T cells and decreasing the infiltration of regulatory T cells. Bevacizumab, paclitaxel and carboplatin regimen have already been reported to have a synergistic effect with anti-PD-1/PD-L1 based immunotherapy in other solid cancers [atezolizumab (anti-PDL1), paclitaxel, carboplatin and bevacizumab in non-small cell lung cancer (NSCLC)].

Based on the significant clinical benefit of Oregovomab with paclitaxel and carboplatin, it is hoped that the addition of bevacizumab will improve response and clinical benefit based on synergistic effects with immunotherapy.

Therefore, there is a need for novel method for use of therapeutic monoclonal antibodies such as Oregovomab and bevacizumab with chemotherapy regimen, such as paclitaxel and carboplatin.

### SUMMARY

According to an embodiment, there is provided a method for improving likelihood of survival in a stage III-IV ovarian cancer patient, the method comprising:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
thereby increasing the patient's likelihood of survival in comparison with a control patient who has been diagnosed with stage III-IV ovarian cancer and has received treatment consisting of 6 cycles of chemotherapy consisting of carboplatin and paclitaxel administration,
wherein in step (a) paclitaxel, carboplatin, and bevacizumab are administered in this order; and in step (b) paclitaxel, mAb-B43.13, carboplatin, and bevacizumab are administered in this order on the same day.

The time interval between every two consecutive cycles of the 6 cycles of chemotherapy may be 1 week, 2 weeks, 3 weeks, or 1 month.

The time interval between every two consecutive cycles of the 6 cycles of chemotherapy may be 3 weeks.

The step (c) may be performed about 12 weeks after cycle 5 of the 6 cycles of chemotherapy.

2 mg of mAb-B43.13 may be administered. 1 mg of mAb-B43.13 may be administered.

The mAb-B43.13 may be administered in a volume of 50 ml by a 20-minute infusion.

175 mg/m² of body surface area paclitaxel may be administered. The paclitaxel may be administered over 3 h.

Area under the curve 5 carboplatin may be administered.

The carboplatin may be administered over 1 h.

The paclitaxel and carboplatin may be administered with an antiemetic medication.

The paclitaxel and carboplatin may be administered with a hypersensitivity medication.

The method may further comprise bevacizumab maintenance therapy.

The time interval between a last cycle of the 6 cycles of chemotherapy and bevacizumab maintenance therapy, or a time interval between each cycle of bevacizumab maintenance therapy is 1 week, 2 weeks, 3 weeks, or 1 month.

The bevacizumab maintenance therapy may be every 3 weeks.

15 mg/kg body weight bevacizumab may be administered.

The bevacizumab may be administered over 30 to 90 minutes.

The bevacizumab may be administered over 30 minutes, or 60 minutes, or 90 minutes.

Each of mAb-B43.13, carboplatin, paclitaxel and bevacizumab may be intravenously administered.

The method may comprises no mAb-B43.13 maintenance therapy.

According to another embodiment, there is provided a chemotherapeutic agent, a therapeutic monoclonal antibody specific for a tumor associated antigen, and a therapeutic monoclonal antibody specific for a vascular endothelial growth factor A (VEGF-A), for use in inhibiting stage III-IV ovarian cancer tumor growth in a patient, wherein the chemotherapeutic agent is a combination of paclitaxel and carboplatin, wherein the therapeutic monoclonal antibody specific for a tumor associated antigen is mAb-B43.13 (oregovomab), wherein the therapeutic monoclonal antibody for VEGF-A is bevacizumab, and wherein paclitaxel, carboplatin, and mAb-B43.13 are administered according to the following schedule:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
wherein in step (a) paclitaxel, carboplatin, and bevacizumab are administered in this order; and
wherein in step (b) paclitaxel, mAb-B43.13, carboplatin, and bevacizumab are administered in this order on the same day.

According to another embodiment, there is provided a use of a chemotherapeutic agent, a therapeutic monoclonal antibody specific for a tumor associated antigen, and a therapeutic monoclonal antibody specific for a vascular endothelial growth factor A (VEGF-A), for inhibiting stage III-IV ovarian cancer tumor growth in a patient, wherein the chemotherapeutic agent is a combination of paclitaxel and carboplatin, wherein the therapeutic monoclonal antibody specific for a tumor associated antigen is mAb-B43.13 (oregovomab), wherein the therapeutic monoclonal antibody for VEGF-A is bevacizumab, and wherein paclitaxel, carboplatin, and mAb-B43.13 are administered according to the following schedule:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
wherein in step (a) paclitaxel, carboplatin, and bevacizumab are administered in this order; and
wherein in step (b) paclitaxel, mAb-B43.13, carboplatin, and bevacizumab are administered in this order on the same day.

According to another embodiment, there is provided a use of a chemotherapeutic agent, a therapeutic monoclonal antibody specific for a tumor associated antigen, and a therapeutic monoclonal antibody specific for a vascular endothelial growth factor A (VEGF-A) in the manufacture of a medicament for inhibiting stage III-IV ovarian cancer tumor growth in a patient, wherein the chemotherapeutic agent is a combination of paclitaxel and carboplatin, wherein the therapeutic monoclonal antibody specific for a tumor associated antigen is mAb-B43.13 (oregovomab), wherein the therapeutic monoclonal antibody for VEGF-A is bevacizumab, and wherein paclitaxel, carboplatin, and mAb-B43.13 are administered according to the following schedule:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
wherein in step (a) paclitaxel, carboplatin, and bevacizumab are administered in this order; and
wherein in step (b) paclitaxel, mAb-B43.13, carboplatin, and bevacizumab are administered in this order on the same day.

The time interval between every two consecutive cycles of the 6 cycles of chemotherapy may be 1 week, 2 weeks, 3 weeks, or 1 month.

The time interval between every two consecutive cycles of the 6 cycles of chemotherapy may be 3 weeks.

The step (c) may be performed about 12 weeks after cycle 5 of the 6 cycles of chemotherapy.

2 mg of mAb-B43.13 may be administered.

1 mg of mAb-B43.13 may be administered.

The mAb-B43.13 may be administered in a volume of 50 ml by a 20-minute infusion.

175 mg/m² of body surface area paclitaxel may be administered.

The paclitaxel may be administered over 3 h.

Area under the curve 5 carboplatin may be administered.

The carboplatin may be administered over 1 h.

The paclitaxel and carboplatin may be administered with an antiemetic medication.

The paclitaxel and carboplatin may be administered with a hypersensitivity medication.

The chemotherapeutic agent and therapeutic monoclonal antibodies, or the use of the present invention may further comprise bevacizumab maintenance therapy.

The time interval between a last cycle of the 6 cycles of chemotherapy and bevacizumab maintenance therapy, or a time interval between each cycle of bevacizumab maintenance therapy may be 1 week, 2 weeks, 3 weeks, or 1 month.

The the bevacizumab maintenance therapy may be every 3 weeks.

15 mg/kg body weight bevacizumab may be administered.

Bevacizumab may be/administered over 30 to 90 minutes.

The bevacizumab may be administered over 30 minutes, or 60 minutes, or 90 minutes.

Each of mAb-B43.13, carboplatin, paclitaxel and bevacizumab may be intravenously administered.

The method may comprise no mAb-B43.13 maintenance therapy.

The following terms are defined below.

The terms “administration of” and/or “administering a” is intended to mean providing an antibody, chemotherapy, and/or their combination according to the present invention to a subject in need of treatment.

The term “composition” intended to mean a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the pharmaceutically acceptable carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing an antibody according to the present invention and a pharmaceutically acceptable carrier. By “pharmaceutically acceptable” it is meant the carrier, diluent or excipient must be compatible with the other ingredients of the formulation and not deleterious to the recipient thereof.

The term “chemotherapy regimen” is intended to mean combination of several chemotherapeutic agents. The rationale behind such chemotherapy regimen is that different chemotherapy drugs work through different cytotoxic mechanisms, and that the results of using multiple drugs will be synergistic to some extent. Because they have different dose-limiting adverse effects, they can be given together at full doses in chemotherapy regimens. Chemotherapy regimen may include induction and maintenance regimen.

The term ‘induction regimen” is intended to mean a chemotherapy regimen used for the initial treatment of a disease.

The term “maintenance regimen” is intended to mean the ongoing use of chemotherapy to reduce the chances of a cancer recurring or to prevent an existing cancer from continuing to grow.

In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the antibody or fragment thereof, preferably in purified form, together with a suitable amount of carrier to provide the form for proper administration to the patient. The formulation should suit the mode of administration.

The terms “inhibit”, “inhibition” or “inhibiting” as used herein in the context of the invention means to slow, hinder, restrain reduce or prevent. For example, “inhibiting growth” of a tumor cell as that term is used herein means to slow, hinder, restrain, reduce or prevent the tumor cell from growing.

The term “administering” as used herein refers to any action that results in exposing or contacting a composition containing the therapeutic monoclonal antibodies of the present invention in combination with the disclosed chemotherapy regimen. As used herein, administering may be conducted in vivo, in vitro, or ex vivo. Particularly, administering is to an ovarian cancer patient, for example a stage III-IV ovarian cancer patient, and more specifically, stage III-IV ovarian cancer patient that are BRCA-wild type that are platinum sensitive. For example, a composition may be administered by injection or through an endoscope. Administering also includes the direct application to cells of a composition according to the present invention. For example, during the course of surgery, tumor cells may be exposed. In accordance with an embodiment of the invention, these exposed cells (or tumors) may be exposed directly to a composition of the present invention, e.g., by washing or irrigating the surgical site and/or the cells, or by direct intra-tumoral injection of the therapeutic monoclonal antibody specific for a tumor associated antigen in combination with at least one immunostimulatory compound, and at least one immune homeostatic checkpoint inhibitor individually or in a mixture.

The term “subject” as used herein, is a human patient or other animal such as another mammal with functional mast cells, basophils, neutrophils, eosinophils, monocytes, macrophages, dendritic cells, and Langerhans cells. In humans, the appropriate cells express the high affinity receptor for IgG for the administered IgG antibody of the invention, as well as IgE (FcεRI) for the administered IgE antibody of the invention. Particularly, the subject may be an ovarian cancer patient, for example a stage III-IV ovarian cancer patient, and more specifically, stage III-IV ovarian cancer patient that are BRCA-wild type that are platinum sensitive.

As used herein, a reduction in growth kinetics, or complete elimination of, a cancer tumor or a metastasized cell or tumor as used herein is defined to mean that which is as understood in the art. For example, a reduction in growth kinetics means a reduction in the exponential growth, specific growth rate, or doubling time of a primary solid tumor, metastasized cell, or metastasized tumor relative to the exponential growth, specific growth rate, or doubling time normally observed in vivo or in vitro fora given tumor type. Complete elimination of a tumor is the absence of tumor presence, either by symptoms, physical exam, or radiographic imaging, in the presence of the therapeutic monoclonal antibody specific for a tumor associated antigen in combination with at least one immunostimulatory compound, and at least one immune homeostatic checkpoint inhibitor, where a tumor was previously seen to be present by these detection methodologies.

The term “time sufficient for treatment” or is intended to mean any period of time suitable to effect treatment with the immune adjuvant (e.g. chemotherapy). In embodiments, that period may be the time of a cycle used in standard to care for the immune adjuvant (e.g. chemotherapy). Examples of standard of care treatments may be found for example in Gynecologic Oncology Group (GOG) Chemotherapy Procedures Manual, incorporated herein by reference. The length of chemotherapy treatment is determined by a variety of factors. These include the type of cancer, the extent of cancer, the types of drugs that are given, as well as the expected toxicities of the drugs and the amount of time necessary to recover from these toxicities. Many chemotherapy treatment schedules (often referred to as Standard of Care (SOC), including the type and length of chemotherapy treatment) have been determined through clinical trials that compared them and determined which had the most benefit and was most well tolerated. In general, chemotherapy treatment is given in cycles. This allows the cancer cells to be attacked at their most vulnerable times and allows the body's normal cells time to recover from the damage. There are really three issues regarding the cycle time, duration of the cycle, frequency of the cycle, and how many cycles. Duration of the cycle: chemotherapy treatment may be a single drug or a combination of drugs. The drugs may all be given on a single day, several consecutive days, or continuously as an outpatient or as an inpatient. Treatment could last minutes, hours, or days, depending on the specific protocol. Frequency of the cycle: chemotherapy may repeat weekly, bi-weekly, or monthly. Usually, a cycle is defined in monthly intervals. For example, two bi-weekly chemotherapy sessions may be classified as one cycle. The number of cycles: in most cases, the number of cycles—or the length of chemotherapy from start to finish—has been determined by research and clinical trials.

When cure is the treatment goal. Adjuvant chemotherapy (therapy after surgery has removed all visible cancer) may last 4-6 months. Adjuvant chemotherapy is common in cancers of the breast and colon. In cancers of the testis, Hodgkin and non-Hodgkin lymphoma, and leukemias, length of chemotherapy treatment may be up to a year. When there is visible disease, the length of chemotherapy treatment will depend upon the response of the disease to therapy. If the disease disappears completely, chemotherapy may continue for 1-2 cycles beyond this observation to maximize the chance of having attacked all microscopic disease. If the disease shrinks but does not disappear, chemotherapy will continue as long as it is tolerated, and the disease does not grow. If the disease grows, the chemotherapy will be stopped. As patients experience toxicities and blood cell counts, the actual timing of the cycles is sometimes delayed according to the necessities of each patient's circumstance. Depending on the health and wishes of the patient, either different drugs may be given to try to kill the cancer, or chemotherapy will be stopped, and the goal changed to focus on patient comfort. In an embodiment, for example, the administration of the immune adjuvant therapy combining paclitaxel and carboplatin with oregovomab and bevacizumab is often performed in cycles of about 21 days (3 weeks).

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates a phase 1 b single arm clinical trial protocol to evaluate the safety and activity of oregovomab, bevacizumab, paclitaxel, and carboplatin according to an embodiment of the present invention.

FIG. 2 illustrates a phase 2 single arm clinical trial protocol to evaluate the safety and activity of oregovomab, bevacizumab (Beva), paclitaxel (Paoli), and carboplatin (Carbo) according to an embodiment of the present invention. CT=Computed tomography (CT) scan.

FIG. 3 illustrates a study duration for a single arm clinical trial protocol according to an embodiment of the present invention.

### DETAILED DESCRIPTION

In embodiments there is disclosed a method for improving likelihood of survival in a stage III-IV ovarian cancer patient, the method comprising:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 (oregovomab) concurrently during cycles 1, 3, and 5 of the 6 cycles of chemotherapy; and
(c) administering to the patient in a final dose of monoclonal antibody mAb-B43.13 (oregovomab) without concurrent chemotherapy about 10 to about 14 weeks after cycle 5 of the 6 cycles of chemotherapy,
thereby increasing the patient's likelihood of survival in comparison with a control patient who has been diagnosed with stage III-IV ovarian cancer and has received treatment consisting of 6 cycles of chemotherapy consisting of carboplatin and paclitaxel administration,
wherein in step (a) paclitaxel, carboplatin, and bevacizumab are administered in this order; and
wherein in step (b) paclitaxel, mAb-B43.13, carboplatin, and bevacizumab are administered in this order on the same day.

According to embodiments, the time interval between every two consecutive cycles of the 6 cycles of chemotherapy is 1 week, 2 weeks, 3 weeks, or 1 month. In a particular embodiment, the time interval between every two consecutive cycles of the 6 cycles of chemotherapy may be about 3 weeks, or about 21 days.

According to embodiments, step c) is performed about 10 to about 14 weeks after cycle 5 of the 6 cycles of chemotherapy, or from about 10 to about 13 weeks, or about 10 to about 12 weeks, or about 10 to about 11 weeks, or about 11 to 14 weeks, or about 11 to 13 weeks, or about 11 to 12 weeks, or about 12 to 14 weeks, or about 12 to 13 weeks, or about 10 weeks, or about 11 weeks, or about 12 weeks, or about 13 weeks, or about 14 weeks, or about 2.5 month, about 3 months, about 3.5 months after cycle 5 of the 6 cycles of chemotherapy.

The present invention is directed to the combination of monoclonal antibodies oregovomab and bevacizumab with paclitaxel and carboplatin to provides for a synergistic effect between these two immune modulators and standard of care chemotherapy in hopes of greatly enhancing patient survival. This is in stark contrast to the use of chemotherapy alone followed by monoclonal antibody oregovomab alone after the initial chemotherapy treatment, which showed no improvements in clinical outcome in advanced ovarian cancer (Berek et al. J Clin Onc 27:418-425, 2009). This is also in contrast with the study of Braly et al. (J Immunother 2009; 32:54-65) which prescribed 8 cycles of chemotherapy including oregovomab immunotherapy at cycles 1, 3, and 5; two additional rounds of chemotherapy and immunotherapy at 12 and 24 weeks past cycle 5, followed by follow-up additional immunotherapy (6 rounds) for up to two years—for a total of 11 doses of oregovomab immunotherapy. The present invention includes a maximum of 6 cycles of paclitaxel and carboplatin chemotherapy, combined with bevacizumab immunotherapy at cycles 1 to 6, and combined with oregovomab immunotherapy at cycles 1, 3 and 5, and a final round of immunotherapy alone at 10 to 14 weeks (preferably 12 weeks) past cycle 5, for a total of 4 rounds of oregovomab immunotherapy. According to an embodiment, there may be no follow-up or maintenance oregovomab immunotherapy. The treatment is believed to improved clinical outcome in advanced ovarian cancer patients. In particular, a direct comparison may be made between the study of Braly et al., where the present invention is believed to display much improved progression-free survival.

### Oregovomab

Oregovomab, also known as mAb-B43.13, is a murine IgG1 antibody specific to CA125 (MUC16). Antibody B43.13 was deposited under the Budapest Treaty in the ATCC, 1801 University Blvd., Manassas, Va. 20110-2209 and given the Patent Deposit Designation PTA-1883 on May 18, 2000. [R. Madiyalakan et al, Hybridoma, 14:199-203 (1995), incorporated herein by reference].

In embodiments, 2 mg of mAb-B43.13 may be administered. According to another embodiment, 1 mg of mAb-B43.13 may be administered. According to another embodiment, mAb-B43.13 may be administered in a volume of 50 ml by a 20-minute infusion.

According to another embodiment, there may be no follow-up or maintenance oregovomab immunotherapy pursuant to the use of the antibody as per the described cycles of administration above.

### Bevacizumab

Bevacizumab, sold under the brand name Avastin™, is a medication used to treat several types of cancers and a specific eye disease. For cancer it is given by slow injection into a vein (intravenous) and used for colon cancer, lung cancer, glioblastoma, ovarian cancer and renal-cell carcinoma.

Common side effects when used for cancer include nose bleeds, headache, high blood pressure, and rash. Other severe side effects include gastrointestinal perforation, bleeding, allergic reactions, blood clots, and an increased risk of infection. Bevacizumab is a monoclonal antibody that functions as an angiogenesis inhibitor. It works by slowing the growth of new blood vessels by inhibiting vascular endothelial growth factor A (VEGF-A), in other words anti-VEGF therapy.

Bevacizumab may be used in combination with chemotherapy for stage III or IV of ovarian cancer after initial surgical operation, followed by single-agent bevacizumab. The approval was based on a study of the addition of bevacizumab to carboplatin and paclitaxel. Progression-free survival was increased to 18 months from 13 months.

Bevacizumab, in combination with carboplatin and paclitaxel is also indicated for the front-line treatment of adults with advanced (International Federation of Gynecology and Obstetrics (FIGO) stages IIIB, IIIC and IV) epithelial ovarian, fallopian tube, or primary peritoneal cancer. Bevacizumab, in combination with carboplatin and gemcitabine or in combination with carboplatin and paclitaxel, is indicated for treatment of adults with first recurrence of platinum-sensitive epithelial ovarian, fallopian tube or primary peritoneal cancer who have not received prior therapy with bevacizumab or other VEGF inhibitors or VEGF receptor-targeted agents.

According to another embodiment, the method of the present invention may further comprise bevacizumab maintenance therapy. During the maintenance phase, bevacizumab may be administered intravenously until disease progression or unacceptably toxicity according to previous combination regimens. For example, the time interval between the last cycle of the 6 cycles of chemotherapy and bevacizumab maintenance therapy, or the time interval between each cycle of bevacizumab maintenance therapy may be 1 week, 2 weeks, 3 weeks, or 1 month. Preferably, the bevacizumab maintenance therapy is every 3 weeks.

During the 6 cycles of chemotherapy, or during the bevacizumab maintenance therapy, 15 mg/kg body weight bevacizumab is administered. The bevacizumab may be administered over 30 to 90 minutes, for example over 30 minutes, or 60 minutes, or 90 minutes.

### Paclitaxel

Paclitaxel is supplied as a sterile solution concentrate. For example at a 6 mg/mL, in 5 mL vials (30 mg/vial) or 17 mL vials (100 mg/vial) in polyoxyethylated castor oil (Cremophor EL) 50% and dehydrated alcohol, USP, 50%. The appropriate dose of paclitaxel may be diluted in 500-1000 mL of 9% Sodium Chloride injection, USP or 5% Dextrose injection, USP (D5W). Paclitaxel may be prepared in glass or polyolefin containers due to leaching of diethylhexylphthalate plasticizer from polyvinyl chloride (PVC) bags and intravenous tubing by the Cremophor vehicle in which paclitaxel is solubilized.

Paclitaxel may be used at a dose of 175 mg/m², and is to be administered via an infusion control device (pump) using non-PVC tubing and connectors, as a 3-hour continuous IV infusion. Due to the risk of immediate hypersensitivity reaction, paclitaxel is the first drug to be infused during any combination.

### Carboplatin

Carboplatin is supplied as a sterile lyophilized powder available in single-dose vials containing 50 mg, 150 mg and 450 mg of carboplatin for administration by IV infusion. Each vial contains equal parts by weight of carboplatin and mannitol.

Immediately prior to use, the contents of each vial may be reconstituted with either sterile water for injection, USP, 5% dextrose in water, or 9% sodium chloride injection, USP, according to the following schedule: 50 mg vial with 5 mL, 150 mg vial with 15 mL and 450 mg vial with 45 mL, all producing a concentration of 10 mg/mL.

The dose of carboplatin may be calculated to reach a target area under the curve (AUC) of concentration×time of 5 according to the Calvert formula using an estimated glomerular filtration rate (GFR) from the Jelliffe formula for creatinine clearance (CrCl).

Calvert Formula: Carboplatin dose (mg)=target AUC×(GFR+25)

For the purposes of the method of the present invention, the GFR is considered to be equivalent to the CrCl. The creatinine clearance was to be estimated by the method of Jelliffe using the following formula:

$$C r C l = 0.9 \times \frac{[ 98 - ( 0.8 ( Age - 20 ) ) ]}{Serum Creatinine}$$

Where: CrCl=estimated creatinine clearance in mL/min; Age=patient's age in years; serum creatinine in mg/dL.

The initial dose of carboplatin may be calculated using GFR. In the absence of new renal obstruction or other renal toxicity (i.e., serum creatinine>1.5×ULN), the dose of carboplatin is not recalculated for subsequent cycles, but is to be subject to dose modification for hematologic criteria and other events.

In patients with an abnormally low serum creatinine, due to reduced protein intake and/or low muscle mass, the creatinine clearance (CrCl) is to be determined from a 24 hour urine collection, rather than a Jelliffe formula.

According to embodiments, the administration of paclitaxel and carboplatin may also involve the administration of antiemetic medication and hypersensitivity medication.

According to another embodiment, there is disclosed a chemotherapeutic agent, a therapeutic monoclonal antibody specific for a tumor associated antigen, and a therapeutic monoclonal antibody specific for a vascular endothelial growth factor A (VEGF-A), for use in inhibiting stage III-IV ovarian cancer tumor growth in a patient, wherein the chemotherapeutic agent is a combination of paclitaxel and carboplatin, wherein the therapeutic monoclonal antibody specific for a tumor associated antigen is mAb-B43.13 (oregovomab), wherein the therapeutic monoclonal antibody for VEGF-A is bevacizumab, and wherein paclitaxel, carboplatin, and mAb-B43.13 are administered according to the following schedule:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
wherein in step (a) paclitaxel, carboplatin, and bevacizumab are administered in this order; and
wherein in step (b) paclitaxel, mAb-B43.13, carboplatin, and bevacizumab are administered in this order on the same day.

According to another embodiment, there is disclosed a use of a chemotherapeutic agent, a therapeutic monoclonal antibody specific for a tumor associated antigen, and a therapeutic monoclonal antibody specific for a vascular endothelial growth factor A (VEGF-A), for inhibiting stage III-IV ovarian cancer tumor growth in a patient, wherein the chemotherapeutic agent is a combination of paclitaxel and carboplatin, wherein the therapeutic monoclonal antibody specific for a tumor associated antigen is mAb-B43.13 (oregovomab), wherein the therapeutic monoclonal antibody for VEGF-A is bevacizumab, and wherein paclitaxel, carboplatin, and mAb-B43.13 are administered according to the following schedule:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
wherein in step (a) paclitaxel, carboplatin, and bevacizumab are administered in this order; and
wherein in step (b) paclitaxel, mAb-B43.13, carboplatin, and bevacizumab are administered in this order on the same day.

According to another embodiment, there is disclosed a use of a chemotherapeutic agent, a therapeutic monoclonal antibody specific for a tumor associated antigen, and a therapeutic monoclonal antibody specific for a vascular endothelial growth factor A (VEGF-A) in the manufacture of a medicament for inhibiting stage III-IV ovarian cancer tumor growth in a patient, wherein the chemotherapeutic agent is a combination of paclitaxel and carboplatin, wherein the therapeutic monoclonal antibody specific for a tumor associated antigen is mAb-B43.13 (oregovomab), wherein the therapeutic monoclonal antibody for VEGF-A is bevacizumab, and wherein paclitaxel, carboplatin, and mAb-B43.13 are administered according to the following schedule:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
wherein in step (a) paclitaxel, carboplatin, and bevacizumab are administered in this order; and
wherein in step (b) paclitaxel, mAb-B43.13, carboplatin, and bevacizumab are administered in this order on the same day.

The present invention will be more readily understood by referring to the following examples which are given to illustrate the invention rather than to limit its scope.

### Example 1

### Study Design

This example is a phase 1b/2, single arm clinical trial design to evaluate the safety and activity of oregovomab and bevacizumab, paclitaxel, carboplatin as a combination strategy, in subjects with BRCA-wild type platinum sensitive recurrent ovarian, tubal and primary peritoneal cancer.

### Screening Period (Up to 28 Days):

After signing informed consent, subjects are screened for eligibility during the period of 28 days immediately prior to starting study drug on Week 1 Day 1 visit. Once all eligibility criteria are met, the subjects are enrolled and initiate chemo-immunotherapy.

Treatment Period (about 30 Weeks):

### Phase 1b Clinical Trial (3+3 Method)

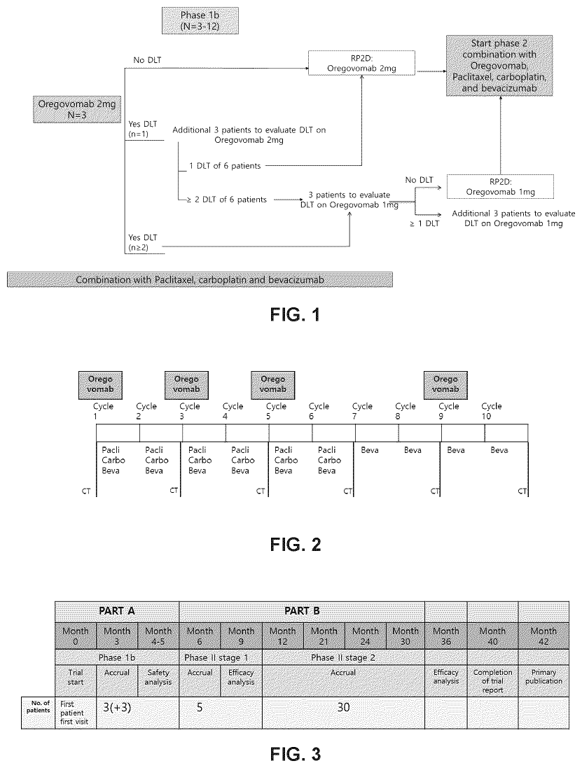

Now referring to FIG. 1. The recommended phase 2 dose (RP2D) of Oregovomab for the evaluation in Phase 2 bevacizumab, paclitaxel and carboplatin combination therapy is selected based on overall safety and tolerability. A 2 mg starting dose of Oregovomab is administered according to previous single and chemotherapy trials. The combination therapy of bevacizumab, paclitaxel, and carboplatin is administered according to standard clinical and institutional practices, as detailed below. For the purposes of this study, oregovomab should be administered after the paclitaxel and before carboplatin in all days. Oregovomab (2 mg) is administered via IV infusion in 50 mL of normal saline over 20 minutes during clinic visits at Day1 of cycle 1. Paclitaxel (175 mg/m² of body surface area) is administered intravenously over 3 h and carboplatin (area under the curve 5) over 1 h with standard antiemetics and hypersensitivity medication for 6 cycles every 3 weeks. Bevacizumab (15 mg/kg body weight) is administered intravenously initially over 90 min. The order of chemotherapy infusion is of concern because of the competition for the antibody clearance pathway between bevacizumab and oregovomab, therefore paclitaxel is infused first followed by oregovomab, which is followed by carboplatin, followed by bevacizumab at the end (paclitaxel oregovomab carboplatin bevacizumab).

The first 3 patients are enrolled and evaluated for the first 21 days for dose-limiting toxicities (DLTs). A DLT is defined as any adverse effect (AE) that is not clearly due to progression of the patient's malignancy, that occurs within the first 21 days of treatment initiation, and that meets at least one of the non-hematologic or hematologic criteria below. Approximately 3 to 12 subjects are enrolled in phase 1b trial (3+3 method). The Sponsor may, after a review of the available safety data (DLT) from subjects who have been on study for 21 days, start a phase 2 trial.

### Non-Hematologic DLT:

≥Grade 3 non-hematologic toxicity according to the Common Toxicity Criteria for Adverse Events (CTCAE) v5 except for the following:
1. Nausea, vomiting, or diarrhea lasting ≤72 h;
2. Grade 3 fatigue lasting ≤7 days;
3. hypersensitivity reactions lasting ≤72 h;
4. Grade 3 hyperglycemia lasting ≤72 h with standard anti-diabetic therapy;
5. Grade 3 increases in liver transaminases in patients with liver metastases. (Note: Grade 4 increases in LFTs in any patient will be considered a DLT);
6. Clinical laboratory abnormalities that are reversible to ≤Grade 1 or baseline status within 72 h with outpatient care and/or monitoring, or that are considered not clinically significant by the Principal Investigator.

### Hematologic DLT:

1. Grade 4 neutropenia [absolute neutrophil count (ANC)<0.5×10⁹/L] prolonged more than 4 days
2. Grade 3 febrile neutropenia (ANC<1.0×10⁹/L with a fever 38.3° C.);
3. Grade 4 thrombocytopenia (<25.0×10⁹/L) lasting >4 days or that requires platelet transfusion prolonged more than 4 days;
4. Grade ≥3 thrombocytopenia associated with Grade ≥3 bleeding;
5. Any hematologic toxicity resulting in death (i.e. Grade 5).

If <1 patient of 3 enrolled patients experiences DLT, enrollment into phase 2 with 2 mg Oregovomab will be initiated. If 1 patient experience a DLT, an additional 3 patients will be enrolled and evaluated for DLTs. If 1 of 6 enrolled patients experiences DLT, enrolment into phase 2 will be initiated, too. If 2 or more patients in the first 3 or second 6 patients experience a DLT, the combination at the current treatment schedule will be considered too acutely toxic and enrollment will begin on a schedule reduced to 1 mg.

Depending on the occurrence rate of DLT, all patients without DLT, who receive the recommended phase 2 treatment regimen, and who are considered evaluable for efficacy from phase 1b will be included in the first stage of phase 2.

### Phase 2 Clinical Trial

Now referring to FIG. 2. Oregovomab (2 mg) will be administered via IV infusion in 50 mL of normal saline over 20 minutes during clinic visits at Day1 of cycle 1, 3, 5, and 12 weeks after cycle 5 (total 4times). Paclitaxel (175 mg/m² of body surface area) is administered intravenously over 3 h and Carboplatin (area under the curve 5) over 1 h with standard antiemetics and hypersensitivity medication for 6 cycles every 3 weeks. Bevacizumab (15 mg/kg body weight) is administered intravenously initially over 90 min (if tolerated, this time is reduced to 60 min; and it could be further reduced to a minimum of 30 min) for 6 cycles every 3 weeks with standard paclitaxel and carboplatin.

In the maintenance phase, bevacizumab (15 mg/kg body weight) is administered intravenously over 60 min every 3 weeks until disease progression or unacceptably toxicity (from cycle 7 to until progression. The order of chemotherapy infusion is of concern because of the neutralizing effect of bevacizumab on Oregovomab, therefore paclitaxel is infused first followed by oregovomab, followed by carboplatin, followed by bevacizumab at the end.

Based on Simon's two stage model, In the first stage of phase 2, 8 patients with the first platinum sensitive recurrent ovarian cancer with BRCA wild type are treated. If at least 7 patients experience an objective response (i.e., partial response or better) by RECIST v1.1, an additional 30 patients are to be enrolled for a total of 38 patients in Part 2. If 7 or fewer patients in the first stage experience an objective response, enrollment into second stage of phase 2 will be discontinued for futility.

For a null hypothesis 18-week Objective Response Rate (ORR18) of 0.75 (response rate of the GOG-0213 trial) and an alternative hypothesis ORR18 of 0.85, the sample size of 38 patients will provide a significance level of approximately 10% and a power of 0.9. If stages 1 and 2 are completed, a minimum of 30 patients must demonstrate a response (PR or better) to reject the null hypothesis. Considering 10% of screening failure rate, overall, 42 patients are enrolled in phase 2 trial. All clinical, safety and immunological evaluations are to be conducted according to a defined schedule during the treatment period and the survival follow-up period.

Imaging studies for disease assessment are done at baseline, Week 6, then every 6 weeks (every 2 cycles) for 36 weeks (until 12^{th} cycle), every 9 weeks for 2 years and every 12 weeks until progression using RECIST v1.1. Subjects are treated until progression, discontinuation due to disease progression, lost to follow-up, withdrawal of consent, treatment with another anti-cancer drug, or death.

Early termination is defined as withdrawal due to intolerance to either of the investigational agents, progression or death. All subjects who are alive and have not withdrawn consent will enter the Post-Treatment Follow-Up Period regardless of reason for discontinuation of treatment.

### Post-Treatment Follow-Up Period:

Initial safety follow-up for 30 days after the last dose of the last treatment taken, followed by Survival Follow-Up.

### Survival Follow Up:

Survival and safety data, including information regarding any interval treatments, are collected every three months [±1 week] for the two years, then every 6 months [±4 weeks] until death, withdrawal of consent, loss to follow-up, or sponsor decision to close the study, whichever comes first for up to 3 years from the end of the treatment period. Follow-up visits for survival may be performed by telephone if a subject is unable to visit the site.

Diagnosis and Main Criteria for Inclusion/Exclusion:

### Main Inclusion Criteria:

1. Adult females (19 years old and older) with CA125-associated first platinum sensitive recurrent epithelial adenocarcinoma of ovarian, fallopian tube or peritoneal origin.
2. Have one of the eligible histologic epithelial cell types: serous adenocarcinoma, endometrioid adenocarcinoma, undifferentiated carcinoma, clear cell adenocarcinoma, mixed epithelial carcinoma, transitional cell carcinoma, malignant Brenner's Tumor, or adenocarcinoma not otherwise specified (N.O.S.).
3. Patients must have had a complete or partial response to front-line platinum-based therapy (at least three cycles) and a treatment-free interval without clinical evidence of progressive disease at least 6 months.
4. Must be platinum sensitive for months.
5. Must have had an elevated serum CA125 >2 times of UNL measured at screening within 28 days of start of study treatment.
6. Must have no known pathogenic significant BRCA 1/2 germline or somatic mutation.
7. Must have measurable disease, including identification of marker lesions, by radiographic or physical criteria suitable for evaluation according to RECIST v1.1 for documentation of disease response or progression.
8. Must have a ECOG Performance Status of 0, 1 or 2.
9. Must have adequate organ function defined as:
   a. neutrophil count ≥1000 μL
   b. hemoglobin >9.0 g/dl
   c. platelet count ≥100,000 μL
   d. Serum creatinine <1.5 times the upper normal limits or Creatinine clearance >45 mL/min/1.73 m²
   e. AST and ALT <2.5 times the upper normal limits
   f. Total bilirubin <1.5 times the upper normal limits
10. Must have voluntarily agreed to participate and have signed the informed consent and are willing to complete all study procedures.

### Main Exclusion Criteria:

1. Patients who have received more than one line of chemotherapy (maintenance is not considered a second line).
2. Have an active autoimmune disease (e.g., rheumatoid arthritis, SLE, ulcerative colitis, Crohn's Disease, MS, ankylosing spondylitis) requiring continuing immune suppressive therapy.
3. Chronically treated with systemic doses of immunosuppressive drugs such as corticosteroids (>10 mg of prednisolone), methotrexate, or immune suppressive monoclonal antibodies.
4. Known allergy to murine proteins or have had a documented anaphylactic reaction to any drug, or a known hypersensitivity to diphenhydramine or other antihistamines of similar chemical structure.
5. Known active hepatitis B virus (HBV) or hepatitis C virus (HCV) infections.
6. Recognized immunodeficiency condition including cellular immunodeficiencies, hypogamma globulinemia or dysgammaglobulinemia; subjects who have acquired, hereditary, or congenital immunodeficiency's, including HIV infection.
7. Have previously received solid organ transplantation.
8. Evidence of any kinds of clinically significant uncontrolled disease such as uncontrolled hypertension, myocardial infarction within 6 months, uncontrolled or unstable angina, congestive heart failure (New York Heart Association Class III or IV).
9. Patients with other invasive malignancies, with the exception of non-melanomatous skin cancer, who had (or have) any evidence of the other cancer present within the last 2 years or whose previous cancer treatment contraindicates with this protocol.
10. Have ever previously received oregovomab and/or bevacizumab.
11. Patients who received major surgical procedure with 28 days (except for any surgery related to ovarian cancer treatment).
   Pregnant or breast-feeding.

Study Objectives and Endpoints:

TABLE 1
Study Objectives and Endpoints

| | |
|---|---|
| Objectives | Endpoints |

Primary

| | |
|---|---|
| Phase 1b: To establish safety | Phase 1b: Incidence |
| and tolerability of oregovomab | of Dose-limiting |
| added to paclitaxel, | toxicities (DLTs) |
| carboplatin and bevacizumab | Phase 2: ORR, defined as the |
| and to identify the | proportion of subjects with |
| recommended phase 2 | CR or PR by RECIST v1.1 |
| treatment regimen | |
| Phase 2: Objective Response Rate | |

Secondary

| | |
|---|---|
| Phase 1b: Objective | Phase 1b: ORR, defined as |
| Response Rate (ORR) | the proportion of subjects |
| by RECIST v1.1, | with CR or PR by RECIST v1.1 |
| Duration of Response (DOR), | Phase 2: |
| Progression-Free Survival (PFS) | Incidence, nature, and intensity |
| and Overall Survival (OS) | of AEs according to CTCAE v5.0. |
| Phase 2: Safety | PFS, defined as date of first |
| and tolerability, | study treatment to the date |
| DOR, PFS, OS | of event defined as the first |
| | documented progression |
| | as per RECIST v1.1 or |
| | death due to any cause. |
| | OS, defined as date of first |
| | study treatment to date of |
| | death due to any cause |

Exploratory

| | |
|---|---|
| Immunological Responses/ | Absolute numbers of FACS |
| Biomarkers | generated CA125 specific |
| | IFNγ+ CD8+ T-cells |
| | NLR associations (high-low subgroup |
| | correlations with clinical outcomes) |
| | Other cellular phenotypes (percentage |
| | and absolute specific memory, |
| | regulatory and B-cell subtypes). |

Study Duration

Subjects are treated for approximately 30 weeks during the Treatment Period, enter Post-Treatment Follow Up Phase with an initial 30-day safety follow-up and then followed for survival until death, withdrawal of consent, lost to follow-up, sponsor decision to close study for up to 3 years after treatment exit or early termination. It is anticipated that recruitment will be completed within approximately 30 months and the entire study, including follow up period, will last approximately 4 years.

### Investigational Product (IP): Oregovomab (MAb-B43.13)

Dosage Form: Lyophilized formulation

Dose: 2 mg, over 20±5 minutes

Mode of Administration/Location: Intravenous/In clinic

Frequency: In Clinic on Day 1 of Week 1, Week 9, Week 15, and Week 27 (see Time and Events Schedule).

Storage: Store un-reconstituted vials at 2° C. to 8° C. Following reconstitution, oregovomab MAb-B43.13 solution may be stored at room temperature (20-25° C.) for up to 4 hours.

### Combination Agent: Paclitaxel

Dosage Form: sterile solution, commercially available formulation.

Dose: 175 mg/BSA for 3 hrs with standard premedication for hypersensitivity.

Mode of Administration/Location: Intravenous/In clinic.

Frequency: Every 3 weeks for 6 cycles.

Storage: Store at 2° C. to 25° C. (36° F. to 77° F.).

Combination Agent: Bevacizumab

Dosage Form: sterile solution, commercially available formulation.

Dose: 15 mg/kg for 90 min first time (if tolerated, this time was reduced to 60 min, and could be further reduced to a minimum of 30 min).

Mode of Administration/Location: Intravenous/In clinic.

Frequency: Every 3 weeks until progression.

Storage: Store at 2° C. to 8°.

### Combination Agent: Carboplatin

Dosage Form: sterile solution, commercially available formulation

Dose: AUC 5

Mode of Administration/Location: Intravenous/In clinic

Frequency: Every 3 weeks for 6 cycles

Storage: Store at protected from light

All combination agents (paclitaxel, carboplatin and bevacizumab) are covered by government insurance as investigator initiated clinical trial supporting program of Korea.

Placebo: Not applicable

### Prior and Concomitant Medications

Any medication (including over the counter or prescription medicines, vitamins, and/or herbal supplements) deemed for supportive care and safety of the subject received at the time of enrollment or receives during the study must be recorded in the eCRF along with reason for use, dates of administration including start and end dates, and dosage information including dose and frequency.

For oregovomab, the use of pre-infusion medication and post-infusion medication is allowed to prevent or treat infusion reactions. Subjects may be pretreated with corticosteroids (such as dexamethasone), diphenhydramine, and H2 antagonists (such as cimetidine or ranitidine).

For paclitaxel, carboplatin and bevacizumab, the use of standard premedication and anti-emetics is allowed.

The subject must notify the investigator about any new medications taken after the start of the study drug. All medications (other than study drug) and significant non-drug therapies (including physical therapy, herbal/natural medications, and blood transfusions) administered during the study must be listed in the eCRF.

Other concomitant medication not listed above may be considered on a case-by-case basis by the investigator in consultation with the Medical Monitor if needed.

Prohibited Concomitant Medications or Medications Taken with Caution

Any concurrent investigational anticancer therapy.

Immunosuppressive medications, including chronic systemic corticosteroids at physiologic doses (equivalent to a dose of 10 mg oral prednisone) 14 days before the first dose (except for participants who require hormone replacement therapy such as hydrocortisone). A temporary course of corticosteroids (i.e., contrast allergy, chronic obstructive pulmonary disease) may be permitted, depending on the duration and dose, after discussion and agreement with the Medical Monitor.

Live attenuated vaccines through 90 days after the last dose of investigational product.

Any concurrent chemotherapy, radiotherapy (except palliative radiotherapy), immunotherapy, biologic, or hormonal treatment. Concurrent use of hormones for noncancer-related conditions is permitted.

### Temporary Discontinuation of Study Treatment(s)

For subjects who do not tolerate the standard of care dose regimens for paclitaxel, carboplatin and bevacizumab, dose adjustments are permitted, as per local prescribing information, to allow the subject to continue the study treatment.

For oregovomab, dose reductions are not permitted. If needed and, at the investigator's discretion, in the case of infusion reactions, oregovomab infusion may be temporarily stopped for up to 2 hours prior to resuming the infusion or the infusion rate may be reduced in accordance with the subject's tolerance. These dose modifications may lower the risk of infusion reactions and may be applied at the discretion of the investigator.
1. During the oregovomab dose interruption, the subject may be provided supportive therapy and monitored for signs and symptoms of allergic reactions or other adverse events.
2. Infusion reactions causing temporary treatment interruption of oregovomab have to recover to ≤CTCAE Grade 1 within 2 hours.
3. Within the 2 hours after temporarily stopping oregovomab, treatment may be re-initiated at lower infusion rate at investigator's discretion.
4. Subjects can be cautiously re-challenged with or without premedication with corticosteroid and/or antihistamine in consultation with the Medical Monitor.
5. If not able to tolerate oregovomab treatment after interrupting treatment or decreasing the rate of infusion, or a re-challenge, the subject will not receive any further treatment with oregovomab. However, the subject may continue being treated with paclitaxel, carboplatin and bevacizumab or physician's choice and be followed for survival.

In the event that there are CTCAE Grade 3≥AEs that occur that cannot be directly attributed to either treatment, the subject must recover to ≤Grade 1 within <7 days, except for the following:
1. Alopecia of any CTCAE grade.
2. Any other skin toxicity recovered to CTCAE Grade ≤2 with adequate supportive care measures within 2 weeks after last dose.
3. Fatigue CTCAE Grade 3 recovered to CTCAE Grade ≤2 within 2 weeks after last dose.
4. Grade 3 AEs of headache, insomnia, diarrhea, nausea and vomiting that resolve to ≤Grade 2 within <72 hours
5. Any other non-hematologic or non-cardiac CTCAE Grade 3 or 4 toxicity that resolved to Grade ≤1 or pre-existing grade at study entry by the next dose.

### Permanent Discontinuation

Subjects may voluntarily discontinue from the study treatment for any reason at any time. The reason should be recorded in the subject's chart and on the appropriate eCRF pages. The following are potential reasons for study treatment discontinuation:
1. Documented Disease Progression, as per RECIST v1.1.
2. Development of a serious or intolerable AE that necessitates discontinuation at the discretion of the Investigator.
3. Pregnancy.
4. Withdrawal of consent by the subject.
5. Loss to follow-up. The site must attempt to contact the subject and reschedule the missed visit as soon as possible and counsel the subject on the importance of maintaining the assigned visit schedule and ascertain whether or not the subject wishes to and/or should continue in the study.
6. Investigator Decision. At the discretion of the Investigator, when he/she believes continued participation is would be detrimental to the subject's well-being.
7. Protocol deviation that, in the opinion of the Sponsor and Investigator, warrants discontinuation from the study.
8. Study Termination by the Sponsor.

Subjects who discontinue study treatment should NOT be considered withdrawn from the study. They should return for the follow-up assessments and be followed for survival. If they fail to return for these assessments for any reason, every effort (e.g., telephone, email, letter) should be made to contact them.

In any subject whose treatment is permanently discontinued, adequate supportive therapy should be continued or initiated as per site standard of care.

### Biomarker Analyses:

Biomarker parameters are analyzed using an explorative approach to determine relationship to study treatment. CA125 specific CD8+ T cells, CD4 T cells and immune profile are analyzed prior to cycle 1 and cycle 3.

### Statistical Methods:

Baseline demographics, disease characteristics, safety parameters, laboratory and clinical data are tabulated by study group using descriptive statistics. Composite Clinical Response are evaluated every 6 weeks and relative to pretreatment baseline using RECIST v1.1 criteria. Kaplan Meier analysis of Progression free survival and overall survival are presented. Correlative analysis of key efficacy parameters are correlated to immune parameters such as neutrophil to lymphocyte ratio at baseline, and presence or emergence of CA125 specific T cell immunity and other defined ovarian cancer risk factors as are detailed in the statistical analysis plan.

### Analysis Populations

The Intent-to-treat (ITT) population includes all subjects who received the first study treatment. The ITT analysis set serves as the primary analysis population for all efficacy endpoints and demographics. The Per-protocol (PP) population includes all subjects who have no major protocol violations that could influence the assessment of efficacy. The PP population are used as supportive for efficacy endpoints. The Safety population includes all subjects who received any amount of study drug (paclitaxel, carboplatin and bevacizumab, or oregovomab). The Safety population are the primary analysis set for safety endpoints.

### Efficacy Analyses

The efficacy analysis are based on the Intent-to-treat Population. Efficacy analyses for binary and time-to-event endpoints are based on programmatically derived response from Investigator-recorded measurements and assessments for target, nontarget, and new lesions according to RECIST v1.1.

The following efficacy endpoints are analyzed for each tumor type/indication:
1. Objective response rate (ORR) is defined as the percentage of participants with a best overall response (BoR) of complete response (CR) or partial response (PR) by confirmation evaluation.
2. Duration of response (DoR) is defined as the time from first documentation of disease response (CR or PR) until first documentation of progressive disease.
3. PFS are measured from the start of treatment until first documentation of progressive disease (according to RECIST v1.1) or death from any cause, whichever occurs first.
4. OS are measured from the start of treatment until death due to any cause.
5. ORR are estimated by the proportion of participants with objective response and their 95% confidence intervals are estimated using the exact binomial method. Time-to-event endpoints (DoR, PFS, and OS) are summarized using the Kaplan-Meier method. Additional supportive analyses of BoR rate, DoR, and PFS are conducted using modified RECIST v1.1, in which a confirmation assessment of progressive disease must be obtained at least 4 weeks after the initial disease assessment indicating progressive disease.

### Safety Analyses

The safety analyses are based on the ITT population. The incidence of AEs and DLTs, as well as changes from baseline in vital signs, clinical laboratory parameters, physical examination findings, ECOG performance status, and ECGs, are analyzed. Summary statistics are provided for treatment-emergent AEs (TEAEs), SAEs, and AE severity and relationship to investigational product(s). The number and percentage of participants in each tumor cohort reporting TEAEs are summarized overall and by the worst grade, system organ class, and preferred term. Similarly, the number and percentage of participants reporting TEAEs considered related to investigational product(s) are summarized. A participant counted once using the highest grade and level of causality if 1 or more occurrences of the same system organ class/preferred term are reported. AEs are graded according to the NCI CTCAE v5.0 and coded using the Medical Dictionary for Regulatory Activities.

### Analysis Timepoints:

Phase 1b: DLT are evaluated 21 days after first 3 patients received first dose of oregovomab, paclitaxel, carboplatin and bevacizumab.

Phase 2: An interim analysis is performed when at least 8 evaluable subjects have completed the Week 18 visit. To be included in the interim analysis group of subjects, the following criteria must be met:
1. Subjects must have had at least 3 doses of oregovomab and at least 80% compliance with paclitaxel, carboplatin and bevacizumab dose administration.
2. Subjects have available data (screening and at least one post-treatment sample collected) for Immunological responses (CA125, etc.)
3. Subjects have completed at least two imaging assessments.

Primary analyses are done after the last subject has had Week 30 visit plus 30-day safety follow-up visit (LPLV). After the last subject exits treatment period, final analyses are completed and Final CSR are completed.

TABLE 2
Clinical Laboratory Tests

| Category | Test Name |
|---|---|
| Hematology: | RBC, hemoglobin, hematocrit, WBC and differential (including |
| | neutrophil, lymphocytes, monocyte, eosinophil, basophil), and platelets |
| Biochemistry: | Glucose, sodium, potassium, chloride, magnesium, calcium, |
| | CO2/bicarbonate, creatinine, AST, ALT, GGT, alkaline phosphatase, |
| | phosphorus, LDH, bilirubin (total and direct), urea (or BUN), total |
| | protein, total cholesterol, albumin, uric acid, lipase, amylase, CPK. In |
| | case of pathological CPK further evaluation (eg, at least by Troponin |
| | assays, CK-MM, CK-MB, ECG exam) must be performed and the |
| | findings documented. |
| | GFR will be estimated by the Cockcroft-Gault formula utilizing serum |
| | creatinine values. |
| Coagulation: | PT, INR, and APTT |
| Urinalysis and Urine | pH, glucose, erythrocytes, leukocytes, protein, color, ketones, nitrite |
| Dipstick | will be analyzed by dipstick. If dipstick is abnormal then perform |
| | microscopic Panel (Red Blood Cells, White Blood Cells, Casts, |
| | Crystals, Bacteria, Epithelial cells); If applicable, urine pregnancy |
| | testing. |
| Thyroid | Screening only: TSH (Thyroid Stimulation Hormone), Free T3 and Free |
| | T. At the subsequent visits as indicated: TSH only; If TSH is abnormal, |
| | test Free T3 and Free T4 |
| Diagnostic | CA-125 |
| Serum Pregnancy Test | hCG test at time points indicated in the Time and Events Schedule. |
| Urine Pregnancy Test | hCG test time points are indicated in the Time and Events Schedule. |
| Exploratory Immune | CA-125 specific CD8 T lymphocytes and CD4 lymphocytes |
| Parameters | Fraction of regulatory T cells, IFN-gamma |

### REFERENCES

1) Ferrandina, G. et al., “A randomized phase II study assessing an optimized schedule of Oregovomab (O) anti-CA125 vaccination with carboplatin paclitaxel (CP) relative to CP alone in front-line treatment of optimally cytoreduced stage III/IV ovarian cancer (EOC).” Journal of Clinical Oncology 35, no. 15 suppl (May 20, 2017) 5536-5536.
2) Madiyalakan, R. et al., “Antiidiotype Induction Therapy: Evidence for the Induction of Immune Response through the Idiotype Network in Patients with Ovarian Cancer after Administration of Anti-CA 125 Murine Monoclonal Antibody B43.13” Hybridoma, 14:199-203 (1995).
3) Ray-Coquard, I. et al., “Olaparib plus Bevacizumab as First-Line Maintenance in Ovarian Cancer.” N Engl J Med 2019; 381:2416-28.
4) Oza, A. M. et al., “Standard chemotherapy with or without bevacizumab for women with newly diagnosed ovarian cancer (ICON7): overall survival results of a phase 3 randomised trial”, Lancet Oncol 2015; 16: 928-36.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While preferred embodiments have been described above and illustrated in the accompanying drawings, it is evident to those skilled in the art that modifications may be made without departing from this disclosure. Such modifications are considered as possible variants comprised in the scope of the disclosure.

1. A method for improving likelihood of survival in a stage III-IV ovarian cancer patient, the method comprising:
(a) administering to a stage III-IV ovarian cancer patient 6 cycles of chemotherapy, wherein in each cycle the patient is administered paclitaxel, carboplatin and bevacizumab on the same day;
(b) administering to the patient monoclonal antibody mAb-B43.13 concurrently during cycles 1, 3, and 5 of the 6 cycles of chemotherapy; and
(c) administering to the patient in a final dose of monoclonal antibody mAb-B43.13 without concurrent chemotherapy about 10 to about 14 weeks after cycle 5 of the 6 cycles of chemotherapy,
thereby increasing the patient's likelihood of survival in comparison with a control patient who has been diagnosed with stage III-IV ovarian cancer and has received treatment consisting of 6 cycles of chemotherapy consisting of carboplatin and paclitaxel administration,
wherein in step (a) paclitaxel, carboplatin, and bevacizumab are administered in this order; and
wherein in step (b) paclitaxel, mAb-B43.13, carboplatin, and bevacizumab are administered in this order on the same day.

2. The method of claim 1, wherein a time interval between every two consecutive cycles of the 6 cycles of chemotherapy is 1 week, 2 weeks, 3 weeks, or 1 month.

3. The method of claim 2, wherein said time interval between every two consecutive cycles of the 6 cycles of chemotherapy is 3 weeks.

4. The method of claim 1, wherein step (c) is performed about 12 weeks after cycle 5 of the 6 cycles of chemotherapy.

5. The method of claim 1, wherein 2 mg of mAb-B43.13 is administered.

6. The method of claim 1, wherein 1 mg of mAb-B43.13 is administered.

7. The method of claim 4, wherein mAb-B43.13 is administered in a volume of 50 ml by a 20-minute infusion.

8. The method of claim 1, wherein 175 mg/m2 of body surface area paclitaxel is administered.

9. The method of claim 8, wherein paclitaxel is administered over 3 h.

10. The method of claim 1, wherein area under the curve 5 carboplatin is administered.

11. The method of claim 10, wherein carboplatin is administered over 1 h.

12. The method of claim 1, wherein paclitaxel and carboplatin is administered with an antiemetic medication.

13. The method of claim 1, wherein paclitaxel and carboplatin is administered with a hypersensitivity medication.

14. The method of claim 1, further comprising bevacizumab maintenance therapy.

15. The method of claim 14, wherein a time interval between a last cycle of the 6 cycles of chemotherapy and bevacizumab maintenance therapy, or a time interval between each cycle of bevacizumab maintenance therapy is 1 week, 2 weeks, 3 weeks, or 1 month.

16. The method of claim 14, wherein said bevacizumab maintenance therapy is every 3 weeks.

17. The method of claim 1, wherein 15 mg/kg body weight bevacizumab is administered.

18. The method of claim 17, wherein bevacizumab is administered over 30 to 90 minutes.

19. (canceled)

20. The method of claim 1, wherein each of mAb-B43.13, carboplatin, paclitaxel and bevacizumab is intravenously administered.

21. The method of claim 1, wherein said method comprises no mAb-B43.13 maintenance therapy.

22.-44. (canceled)
